(19) United States
(12) Patent Application Publication
(10) Pub. No.: US 2025/0138643 A1
(43) Pub. Date: May 1, 2025

(54) SPATIALLY TRACKING MUSCLE ACTIVITY

(71) Applicant: Microsoft Technology Licensing, LLC, Redmond, WA (US)

(72) Inventors: Ryan CHANG, Bellevue, WA (US); Young Soo KIM, Bellevue, WA (US); Kelly A OHM, Seattle, WA (US); Jazmine HOYLE, Kirkland, WA (US); Michael BOHAN, Maple Valley, WA (US); Aditha May ADAMS, Seattle, WA (US); Timothy G ESCOLIN, Seattle, WA (US); Spencer Lee DAVIS, Columbus, OH (US); Scott D SCHENONE, Seattle, WA (US); Eduardo SONNINO, Seattle, WA (US)

(73) Assignee: Microsoft Technology Licensing, LLC, Redmond, WA (US)

(21) Appl. No.: 18/730,592

(22) PCT Filed: Feb. 23, 2023
(86) PCT No.: PCT/US2023/063108

§ 371 Date: Jul. 19, 2024

(30) Foreign Application Priority Data
Feb. 24, 2022 (NL) 2031070

(51) Int. Cl. G06F 3/01; G06F 3/0482

(52) U.S. Cl. CPC G06F 3/017; G06F 3/011; G06F 3/0482

(57) ABSTRACT

A computer-implemented method for spatially tracking muscle activity is disclosed. A muscle activation signal is received from a muscle activation sensor. The muscle activation signal indicates an amount of muscle activation of a muscle associated with a body part. A spatial signal is received from a spatial sensor. The spatial signal indicates a location of the body part in a physical space. Activation data is data that spatially correlates the amount of muscle activation of the body part to the location of the body part in the physical space.

### BACKGROUND

A graphical user interface (GUI) includes a plurality of user interface objects that a user interacts with as part of a personal computing experience. For example, a user may provide user input to select a user interface object as part of performing a computing task. In some implementations, a user may provide user input to interact with a user interface object in a GUI using one or more user-input devices, such as a keyboard, mouse, touch screen, or game controller. In some implementations, a user may provide natural user input to interact with a user interface object in a GUI. For example, a user may perform various gestures that may be recognized by natural user input componentry, such as an infrared, color, stereoscopic, and/or depth camera.

### SUMMARY

A computer-implemented method for spatially tracking muscle activity is disclosed. A muscle activation signal is received from a muscle activation sensor. The muscle activation signal indicates an amount of muscle activation of a muscle associated with a body part. A spatial signal is received from a spatial sensor. The spatial signal indicates a location of the body part in a physical space. Activation data is data that spatially correlates the amount of muscle activation of the body part to the location of the body part in the physical space.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter. Furthermore, the claimed subject matter is not limited to implementations that solve any or all disadvantages noted in any part of this disclosure.

### BRIEF DESCRIPTION OF THE DRAWINGS

FIG. 1 shows different example scenarios of users interacting with graphical user interfaces (GUIs) of different types of computing devices.

FIG. 2 shows an example computing system configured to spatially track muscle activity.

FIG. 3 shows an example muscle activation signal.

FIG. 4 shows an example scenario in which a user's thumb is interacting with a 2D GUI of a smartphone to generate activation data.

FIG. 5 shows an example heat map data structure that correlates an amount of muscle activation of a body part to a location of the body part in a physical space.

FIG. 6 shows an example two-dimensional (2D) GUI including a plurality of user interface objects that are optimized based at least on activation data.

FIG. 7 shows an example three-dimensional (3D) GUI including a non-optimized arrangement of a plurality of user interface objects.

FIG. 8 shows an example 3D GUI including an optimized arrangement of a plurality of user interface objects.

FIGS. 9-10 show an example method of spatially tracking muscle activity.

FIG. 11 shows an example computing system.

### DETAILED DESCRIPTION

Efficiency of user movement defined in terms of muscle activation may be difficult to assess accurately. Further, optimizing a GUI to improve efficiency of user movement when a user is interacting with the GUI is difficult. Previous approaches of optimizing a GUI have employed performance-based metrics. In one example approach, user interface objects are arranged in a GUI based on error rates of touching a designated user interface object (e.g., a virtual button). In another example approach, user interface objects are arranged in a GUI based on movement times to touch a designated user interface object. However, these optimization approaches do not consider a user's muscle state, degree of muscle activation (i.e., strain of the muscle or muscle effort), or a degree of muscle fatigue.

Accordingly, the present description is directed to a computer-implemented method for spatially tracking a user's muscle activity. Such an approach includes receiving a muscle activation signal from a muscle activation sensor. The muscle activation signal from a particular sensor indicates an amount of muscle activation of a muscle associated with a body part. For example, the muscle activation sensor may be used to measure a degree of muscle activation for a particular body part including appendages, such has arms, hands, fingers, or any other muscles. The computer-implemented method further includes receiving a spatial signal from a spatial sensor. The spatial signal from a particular sensor indicates a location of the body part in a physical space. Activation data is generated based at least on the muscle activation signal and the spatial signal. The activation data spatially correlates the amount of muscle activation of the body part to the location of the body part in the physical space. Such activation data can be used to provide an accurate assessment of movement efficiency of the body part in the physical space. For example, determinations of an interaction distance of a user's body part, a range of movement of the user's body part, and degrees of muscle activation/strain/effort of the user's body part as the user's body part moves through the range of motion can be made based at least on the activation data. This analysis can be performed for one or more body parts of one or more users—e.g., analyze muscle strain/effort of a single thumb muscle for a single user, or analyze differences in various muscle strains/efforts of different muscles across different users. This analysis may be performed in a research setting, for example to determine an optimal GUI arrangement for a population of users; and/or this analysis may be performed in a use setting to dynamically adapt a user interface based at least on real-time muscle strain/effort assessments.

The activation data can be leveraged to optimize an arrangement of user interface objects in a GUI in terms of minimizing muscle activation/strain/effort, joint strain, and/or improving movement efficiency. As used herein, “optimized” means decreasing the negative physical consequences of a body movement, but does not necessarily mean maximally decreasing the negative physical consequences, because other considerations may be deemed more important for user satisfaction (e.g., not cluttering all GUI buttons to occupy only 5% of the display area). In one example, a plurality of user interface objects can be arranged in a GUI based at least on the activation data to minimize a degree of muscle activation of the user's muscles when interacting with the GUI. By optimizing an arrangement of user interface objects in a GUI based at least on the activation data, in the short-term, a user's muscle activation/strain/effort may be reduced while interacting with the GUI relative to interacting with another GUI arranged in a different manner that does not consider muscle activation. Moreover, a likelihood of long-term overuse injuries from interacting with the GUI may be reduced relative to interacting with another GUI arranged in a different manner that does not consider muscle activation. Further, optimizing the GUI in terms of user movement efficiency based at least on the activation data may increase user productivity, since a user can make low-strain movements more quickly and more frequently with less fatigue relative to interacting with other GUIs that are arranged using other methods that do not consider minimizing muscle activation/strain/effort.

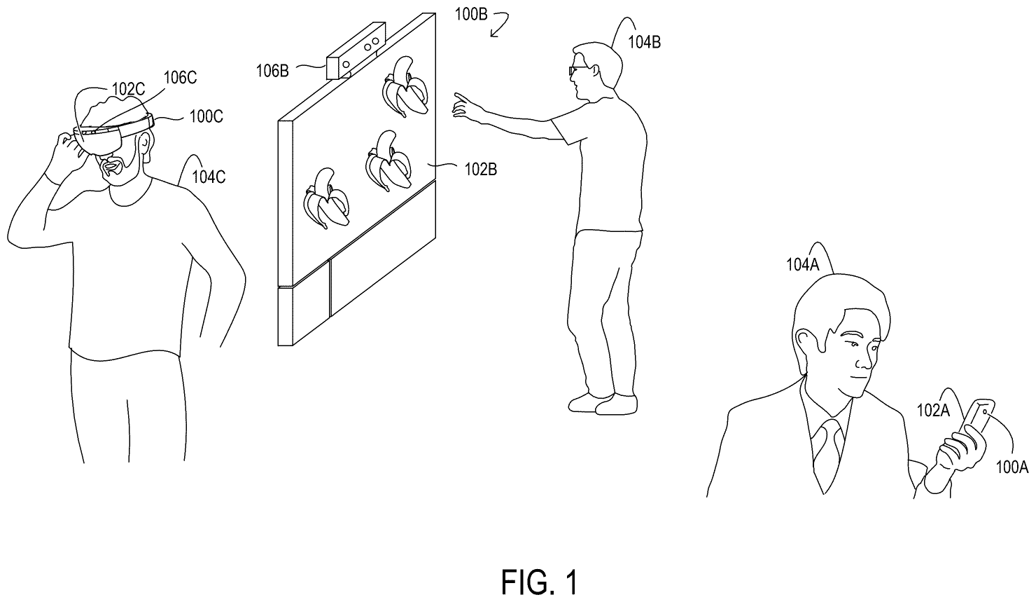

FIG. 1 shows different examples of computing devices (100A-100C) that are configured to present a GUI that can be optimized to minimize muscle activation/strain/effort of a user that is providing user input to interact with the GUI. Device 100A is a smartphone that includes a touch-sensitive display 102A that is configured to visually present a GUI including a plurality of user interface objects. A user 104A provides touch input to the touch-sensitive display 102A to interact with the user interface objects in the GUI. The plurality of user interface objects may be arranged in the GUI based at least on activation data that spatially correlates an amount of muscle activation of the user's fingers to the location of the user's fingers while interacting with the GUI. Device 100B is a video game system that includes a large-format display 102B that is configured to visually present a GUI including a plurality of user interface objects. A user 104B provides natural user input gestures that are captured by a peripheral camera 106B to interact with the user interface objects in the GUI. Device 100C is an augmented-reality headset that includes a 3D display 102C configured to visually present a GUI including a plurality of user interface objects that appear to be incorporated into a physical space surrounding the user 104C. The augmented-reality headset 100C includes an outward-facing camera 106C that is configured to detect natural user input of the user 104C, such as hand gestures, that the user 104C provides to interact with the user interface objects in the GUI.

In each of the examples of the computing devices 100A-100C, the plurality of user interface objects can be arranged in the GUI based at least on activation data that spatially correlates the amount of muscle activation of a body part to a location of the body part in physical space. For example, the plurality of user interface objects can be arranged to reduce or minimize muscle activation (and/or joint strain) of the different body parts while interacting with the GUI. In the case of the smartphone 100A, the plurality of user interface objects can be arranged to reduce or minimize muscle activation of the fingers of the user 104A. In the case of the video game system 100B, the plurality of user interface objects can be arranged to reduce or minimize muscle activation of fingers, hands, arms, shoulders, and legs of the user 104B. In the case of the augmented-reality headset 100C, the plurality of user interface objects can be arranged to reduce or minimize muscle activation of the fingers, hands, and arms of the user 104C. In each example, the GUI may be optimized to reduce or minimize muscle activation of any suitable body part of the user while the user is interacting with the GUI. Such a reduction or minimization of muscle activation while interacting with the GUI improves human-computer interaction and reduces the burden of user input to a computing device, since a user can make low-strain movements more quickly and more frequently with less fatigue relative to interacting with other GUIs that are arranged using other methods that do not consider reducing muscle activation/strain/effort.

In some implementations, such activation data may be used in a research and development scenario. For example, activation data may be aggregated for a plurality of users having different physical characteristics (e.g., different hand size, arm size, leg size) in order to design an arrangement of a plurality of user interface objects in a GUI that is optimized collectively for all of the plurality of users. In some implementations, different optimizations may be found for different subsets of users (e.g., small hands, average hands, big hands). In some implementations, activation data may be generated for a designated user, and an arrangement of a plurality of user interface objects in a GUI may be dynamically adjusted or customized based at least on the user-specific activation data generated for the designated user.

The computing devices 100A-100C are provided as non-limiting examples that may be configured to visually present a GUI including an arrangement of user interface objects that is optimized in terms of minimizing muscle activation/strain/effort, joint strain, and/or improving efficiency of movement of body parts of a user that is interacting with the GUI. The arrangement of user interface objects may be set and/or dynamically changed based at least on activation data that spatially correlates the amount of muscle activation of a body part to a location of the body part in physical space. The concepts discussed herein may be broadly applicable to any suitable type of computing system.

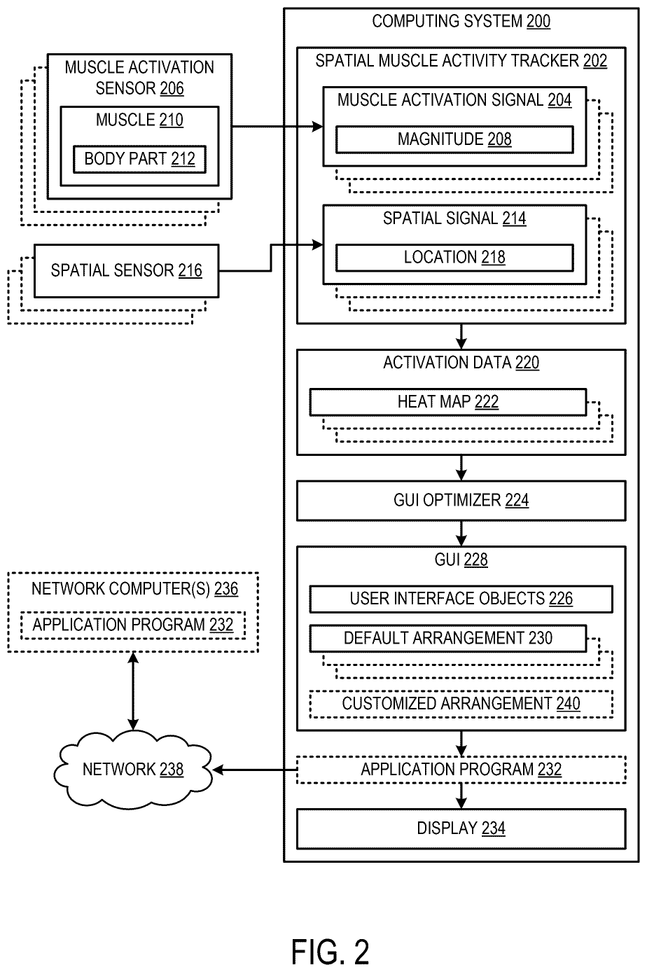

FIG. 2 shows an example computing system 200 that is configured to spatially track muscle activity for GUI optimization. The computing system 200 includes a spatial muscle activity tracker 202 that is configured to generate activation data that spatially correlates an amount of muscle activation of a body part to a location of the body part in physical space. The spatial muscle activity tracker 202 is configured receive one or more muscle activation signals 204 from one or more muscle activation sensors 206. Each muscle activation signal 204 indicates an amount (i.e., magnitude) 208 of muscle activation of a muscle 210 associated with a body part 212 as measured by the corresponding muscle activation sensor 206. In some examples, two or more muscle activation sensors 206 may be cooperatively used to determine an amount of muscle activation of a body part. In some examples, different muscle activation sensors 206 may be used to measure muscle activations of different muscles.

The muscle activation sensor 206 may be configured to measure muscle activation of any suitable muscle corresponding to any suitable body part of a user. For example, the muscle activation sensor 206 may be placed or otherwise focused on a user's hand, wrist, arm, shoulders, head, back, legs, or feet to measure muscle activation of muscles in those body parts.

The muscle activation sensor 206 may take any suitable form of sensor that measures an amount of muscle activation of the muscle 210 associated with the body part 212. In some examples, the muscle activation sensor 206 includes an electromyography (EMG) sensor. In some examples, the muscle activation sensor 206 includes a mechanical expansion sensor that measures muscle activation based at least on muscle expansion. In some examples, the muscle activation sensor 206 includes an optical sensor that can be used to infer muscle activation via image processing. In some implementations, alternatively or in addition, the sensor 210 may be configured to determine an amount of joint strain. For example, an infrared camera may be configured to determine an amount of heating/swelling in or near a user's joints (e.g., thumb, finger, shoulder, elbow, hip, knee, ankle). The sensor 210 may be configured to determine an amount of joint strain in a user's joint in any suitable manner.

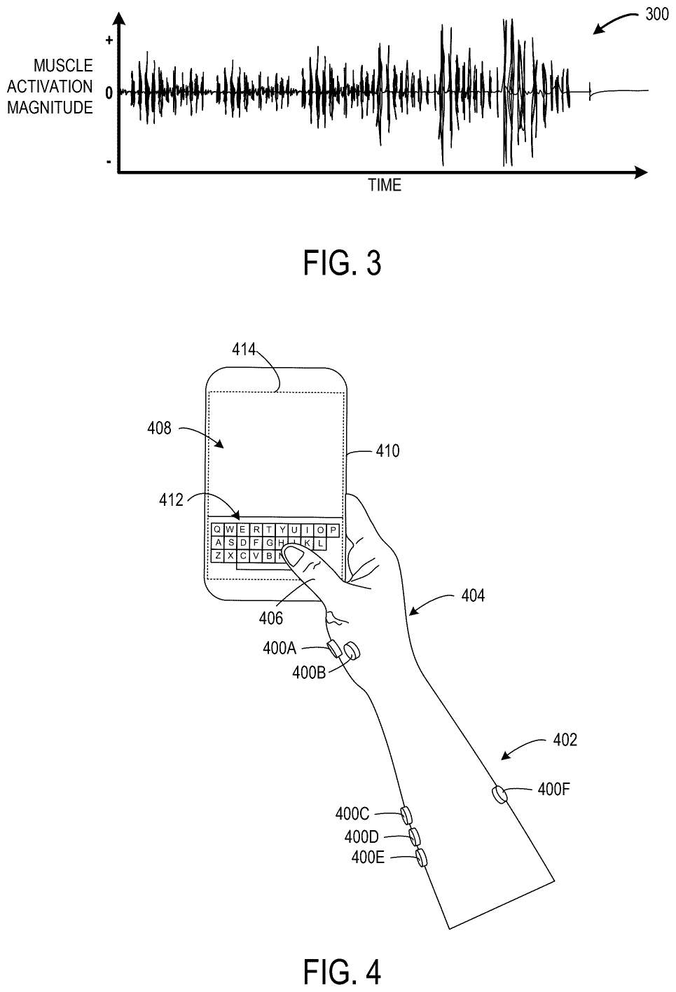

FIG. 3 shows a hypothetical example of a muscle activation signal 300 that could be output from an EMG sensor over a period of time. The muscle activation signal 300 is centered around a baseline (i.e., zero). The baseline indicates when the muscle is in a state of rest where the muscle is not activated. When the muscle activates, the degree of muscle activation is indicated by the magnitude of the muscle activation signal 300. A greater amount of muscle activation or muscle strain/effort corresponds to a greater magnitude of the muscle activation signal 300.

Returning to FIG. 2, the spatial muscle activity tracker 202 is configured to receive one or more spatial signals 214 from one or more spatial sensors 216. Each spatial signal 214 indicates a location 218 of the body part 212 in a physical space. In some examples, the spatial sensor 216 is configured to determine a two-dimensional (2D) location 218 of the body part 212 in a physical space. In one example, the spatial sensor 216 is a touch sensor of a touch-sensitive display device that is configured to determine a 2D location of a finger relative to the touch-sensitive display device. In some examples, the spatial sensor 216 is configured to determine a three-dimensional (3D) location of the body part 212 in a physical space. In one example, the spatial sensor 216 is configured to determine a 6 degrees of freedom (6DOF) location of the body part 212.

The spatial sensor 216 may take any suitable form of sensor that determines a location of the body part 212 in a physical space. In some examples, the spatial sensor 216 includes a camera, such as a RGB, infrared, or depth camera. In such an example, the spatial signal 214 may include images output from the camera that the spatial muscle activity tracker 202 may use to determine the location of the body part 212. In some examples, the spatial sensor 216 includes an inertial measurement unit (IMU) that includes one or more accelerometers and/or one or more gyroscopes, and the spatial muscle activity tracker 202 uses signals output from the IMU to determine the location of the body part 212. In some examples, the spatial sensor 214 includes a touch sensor, such as a trackpad or a touch-sensitive display.

In some implementations, spatial signals from two or more spatial sensors 216 may be cooperatively used to determine a location of the body part 212 in a physical space. In some examples, one or more spatial sensors 206 may be used to determine locations of different body parts. In one example, a camera and an IMU may be cooperatively used in conjunction to determine a location of the body part 212. In another example, a plurality of cameras is used to track movement of the body part 212 in a physical space. In some examples, the plurality of cameras may be incorporated into a wearable device, such as an augmented-reality headset. In other examples, the plurality of cameras could be placed in fixed locations in the physical space to track movement of a user's body parts as the user moves throughout the physical space. Any suitable number of spatial sensors may be used to determine the location of the body part(s) 212 in the physical space.

The spatial muscle activity tracker 202 is configured to output activation data 220 spatially correlating the amount 208 of muscle activation of a particular body part 212 to the location 218 of that particular body part 212. For example, the muscle activation measured at a particular time may be correlated to the body part location determined at that same time, thus allowing the muscle activation at that particular time to be correlated to the body part location at that particular time. This can be replicated for any number of body parts, correlating muscle activation for any number of different muscles with corresponding locations of the relevant body parts. In some examples, the activation data 220 may be arranged in a data structure that correlates an amount of muscle activation to a location for each tracked body part. The activation data 220 may be arranged in any suitable type of data structure.

In some implementations, the activation data 220 may spatially track muscle activation of the body part(s) 212 over a designated period of time. For example, the muscle activations measured at a plurality of different specific times may be correlated to the body part locations determined at those specific times, thus allowing the muscle activations at those specific times to be correlated to the body part locations at those specific times. In some implementations, the spatial muscle activity tracker 202 may be configured to determine an amount of muscle fatigue of the body part 212 based at least on the activation data 220 tracked over the designated period of time. For example, a Fourier transform, or another suitable analysis may be applied to the activation data to determine muscle fatigue.

In some implementations, the spatial muscle activity tracker 202 may be configured to spatially track muscle activity for a plurality of body parts of the same user. For example, a plurality of different muscle activation sensors may be placed on different muscles corresponding to different body parts of the same user. The spatial muscle activity tracker 202 may be configured to receive, from the plurality of muscle activation sensors, a plurality of muscle activation signals corresponding to the plurality of muscles associated with the plurality of body parts of the same user. Each muscle activation signal indicates an amount of muscle activation of the corresponding muscle of the plurality of muscles. Further, the spatial muscle activity tracker 202 may be configured to receive, from one or more spatial sensors, one or more spatial signals indicating locations of the plurality of body parts in a physical space. The spatial muscle activity tracker 202 may be configured to output activation data 220 that spatially correlates the amount of muscle activation of each of the plurality of body parts to locations of each of the plurality of body parts in the physical space. In some implementations, such activation data 220 may be used to optimize a GUI in terms of minimizing muscle activation (and/or joint strain) of the plurality of body parts of the user.

In some implementations, the spatial muscle activity tracker 202 may be configured to spatially track muscle activity for a plurality of instances of the same body part of different users. For example, a plurality of muscle activation sensors may be placed on the same muscle of a plurality of different users. The spatial muscle activity tracker 202 may be configured to receive, from the plurality of muscle activation sensors associated with the different users, a plurality of muscle activation signals. Each muscle activation signal may indicate an amount of muscle activation of the muscle of the corresponding user. The spatial muscle activity tracker 202 may be configured to receive, from one or more spatial sensors associated with the different users, a plurality of spatial signals indicating locations of the plurality of body parts of the plurality of different users. The spatial muscle activity tracker 202 may be configured to output activation data 220 that spatially correlates the amount of muscle activation of each of the plurality body parts of the plurality of different users to each of the locations of the plurality of body parts. By aggregating such activation data 220 for a plurality of different users, spatial muscle activation may be tracked and compared across a population of users. Further, in some implementations, such activation data 220 may be used to optimize a GUI in terms of reducing or minimizing muscle activation (and/or joint strain) for the population of users. For example, in the case of a GUI including a virtual keyboard, the virtual keys may be sized and/or positioned to reduce muscle activation for users that have small hands vs. users that have large hands based on activation data collected from both types of users.

In some implementations, the spatial muscle activity tracker 202 is configured to output the activation data 220 as a heat map data structure 222 that indicates different amounts of muscle activation 208 of the muscle 210 corresponding to the body part 212 at different locations 218 in the physical space. The heat map data structure 222 distinguishes between different amounts of muscle activation that the muscle 210 corresponding to the body part 212 goes through as the body part 212 moves to different locations. In some examples, the activation data 220 may include a plurality of heat maps corresponding to the plurality of different body parts of the same user. In some examples, the activation data 220 may include a plurality of heat maps corresponding to the same body part of the plurality of different users. Such activation data 220 may be aggregated for a plurality of different body parts of the plurality of different users. By using heat map(s) to convey the correlation of muscle activation/strain/effort to locations in space of body part(s), the movement of the body part(s) that cause various degrees of muscle activation/strain/effort can be easily identified and distinguished from each other. Thus, heat map(s) act as tools to enable GUI optimization in terms of reducing or minimizing muscle activation/strain/effort of body part(s) while interacting with the GUI.

In some implementations, the heat map data structure 222 spatially correlates the amount 208 of muscle activation of the body part 212 to 2D locations in physical space. In other implementations, the heat map data structure 222 spatially correlates the amount 208 of muscle activation of the body part 212 to 3D locations in physical space.

FIG. 4 shows an example scenario in which a user's muscle activity is spatially tracked while the user is interacting with a GUI of a smartphone. A plurality of muscle activation sensors 400 (e.g., 400A, 400B, 400C, 400D, 400E, 400F) are arranged on different muscles in a user's arm 402 and hand 404. Each of the muscle activation sensors 400 outputs a muscle activation signal that indicates an amount of muscle activation of a corresponding muscle. Muscle activation signals output by the plurality of muscle activation sensors 400 are used to measure an amount of muscle activation of the user's thumb 406 while the user's thumb 406 provides touch input to a GUI 408 of a smartphone 410. The GUI 408 includes a plurality of user interface objects in the form of virtual keys 412 arranged in a keyboard. 2D locations of the user's thumb 406 are tracked by a touch-sensor 414 of the smartphone 410 as the user's thumb 406 provides touch input to select the different virtual keys 412 of the virtual keyboard. In this example, the touch sensor 414 acts as a spatial sensor that tracks 2D locations of the user's thumb 406 on the touch sensor 414 as a spatial signal. The muscle activation signals from the muscle activation sensors 400 and the spatial signal from the touch sensor 414 can be time synchronized and used to generate activation data that spatially correlates the amount of muscle activation of the user's thumb 406 to the location of the user's thumb 406 while providing user input to the touch sensor 414. In this example, the touch sensor 414 assumes dual roles of sensing touch input to control operation of the smartphone, while also spatially tracking the location of the user's thumb for the generation of activation data. Such dual roles assumed by the touch sensor alleviate the requirement for an additional discrete spatial sensor. Although, in some implementations, the user's thumb may be tracked by a plurality of spatial sensors (e.g., the touch sensor and a camera on the smartphone).

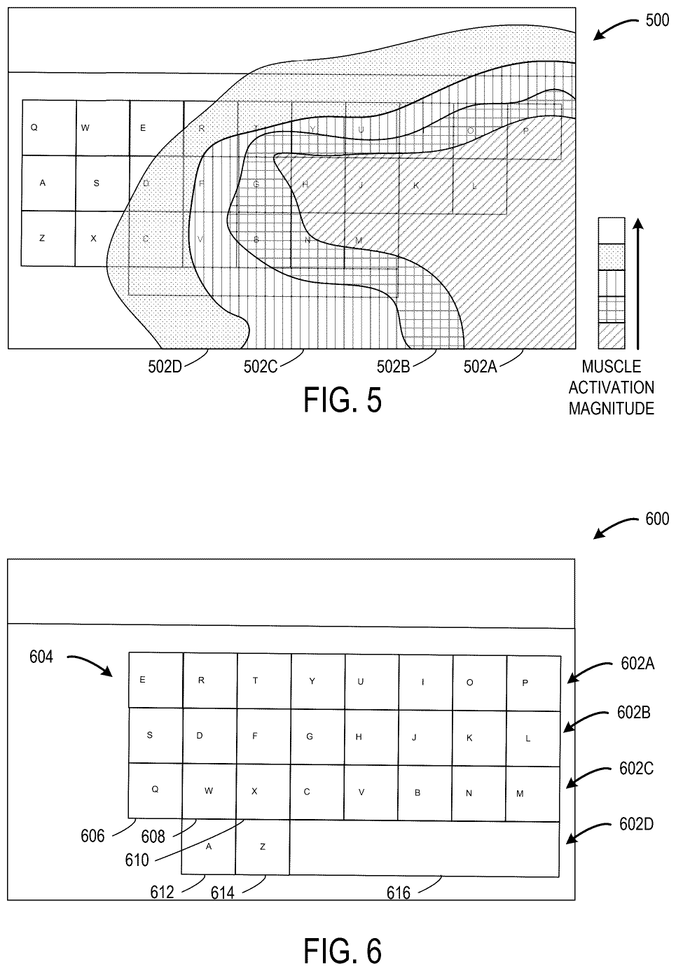

FIG. 5 shows an example hypothetical heat map 500 that could be generated based at least on the muscle activation signals from the muscle activation sensors 400 and the spatial signal from the touch sensor 414 shown in FIG. 4. For example, the heat map 500 corresponds to the heat map 222 shown in FIG. 2. Different activation regions 502 (e.g., 502A, 502B, 502C, 502D) of the heat map 500 indicate different magnitudes of muscle activation exerted by the user's thumb 406 while interacting with the different virtual keys 412 in the GUI. For example, the user's thumb exerts a higher degree of muscle activation to touch virtual keys in a high-activation region 502D than virtual keys in a low-activation region 502A.

The heat map 500 is provided as a non-limiting example. A heat map may include any suitable number of different degrees of magnitude of muscle activation.

In some implementations, the smartphone 410 may be configured to generate the activation data, including the heat map 500, based at least on interaction of the user's thumb 406 with the GUI 408 shown in FIG. 4. In other implementations, the muscle activation signals and the spatial signal may be sent to a separate computing system, such as the computing system 200 shown in FIG. 2, to generate the activation data including the heat map 500.

Returning to FIG. 2, the spatial muscle activity tracker 202 is configured to output the activation data 220 to a GUI optimizer 224. The GUI optimizer 224 is configured to arrange a plurality of user interface objects 226 in a GUI 228 based at least on the activation data 220.

In some examples, the GUI optimizer 224 may set a position of a user interface object of the plurality of user interface objects 226 based at least on the activation data 220. The GUI optimizer 224 may be configured to position more-frequently-used user interface objects having higher interaction frequencies with the body part 212 at locations correlated with a smaller amount of muscle activation based at least on the activation data 220. Further, the GUI optimizer 224 may be configured to position less-frequently-used user interface objects having lower interaction frequencies with the body part 212 at locations correlated with a larger amount of muscle activation based at least on the activation data 220. By arranging the more-frequently-used user interface objects in portions of the GUI where a user exerts less muscle activation/strain/effort, an amount of muscle activation exerted to interact with the more-frequently-used user interface objects may be reduced relative to a GUI that is arranged in a different manner.

In some examples, the GUI optimizer 224 may set a size of a user interface object of the plurality of user interface objects 226 based at least on the activation data 220. For example, the spatial muscle activity tracker 202 may be configured to track interactions of one or more body parts 212 with the plurality of user interface objects 226. Such tracking may be indicated in the activation data 220. The GUI optimizer 224 may be configured to make more-frequently-used user interface objects having higher interaction frequencies with the body part(s) 212 larger in size relative to less-frequently-used user interface objects having lower interaction frequencies. In one example, in the case of a virtual keyboard, virtual keys of letters that are used more frequently may be set to be larger than virtual keys of letters that are used less frequently. In this way, the more-frequently-used virtual keys can be easier to touch accurately. In another example, the space bar virtual key is used more frequently than the quote mark virtual key, so the size of the space bar key may be set larger than the size of the quote mark virtual key. By making the more-frequently-used user interface objects larger in size and the less-frequently-used user interface object smaller in size, an overall amount of space occupied by the total number of user interface objects may remain the same while making the more-frequently-used user interface objects easier to interact with relative to a GUI that is arranged in a different manner.

In the above-described examples, the GUI optimizer 224 is configured to optimize the GUI 228 to minimize muscle activation (and/or joint strain) of a user's body part(s) while the user is interacting with the GUI 228. In some implementations, the GUI optimizer 224 may be configured to optimize the GUI 228 to cause a user to increase a degree of muscle activation/strain/effort exerted by the user while interacting with the GUI 228. For example, the GUI 228 may be optimized in this manner in scenarios where a user desires to strengthen or rehabilitate the muscle(s) (and/or joint(s)) in the body part(s). In some implementations, the GUI optimizer 224 may be configured to arrange the layout of the GUI 228 to increase or maximize muscle activation/strain/effort of a user's body part(s) while the user is interacting with the GUI 228. For example, such a GUI may be incorporated into a video game or a physical exercise/workout computer application program.

In some implementations, the GUI optimizer 224 may optimize a 2D GUI including a plurality of user interface objects each having 2D locations based at least on the activation data 220. FIG. 6 shows an example 2D GUI 600 that is optimized based at least on the heat map 500 shown in FIG. 5. A plurality of rows 602 (e.g., 602A, 602B, 602C, 602D) of the virtual keyboard 604 are shifted to the right side of the GUI 600, such that more of the virtual keys reside in the low-activation region (i.e., region 502A of the heat map 500 shown in FIG. 5) of the GUI 600. Additionally, a Q-key 606, a W-key 608, an X-key 610, an A-key 612, and a Z-key 614 are moved to different rows that cause the keys to be shifted rightward to lower activation regions of the GUI 600 so that the user's thumb may more easily reach these keys when interacting with the GUI 600. Further still, the space bar 616 is increased in size since the space bar 616 may be frequently interacted with when typing out phrases and sentences on the virtual keyboard 604.

The virtual keyboard 604 in the GUI 600 may be optimized to reduce muscle activation of the user's thumb while interacting with the virtual keyboard 604 relative to the virtual keys 412 of the virtual keyboard shown in FIG. 4. The GUI 600 is provided as non-limiting example. A 2D GUI may be optimized in any suitable manner based at least on activation data that spatially correlates an amount of muscle activation of a body part to a location of the body part in a physical space.

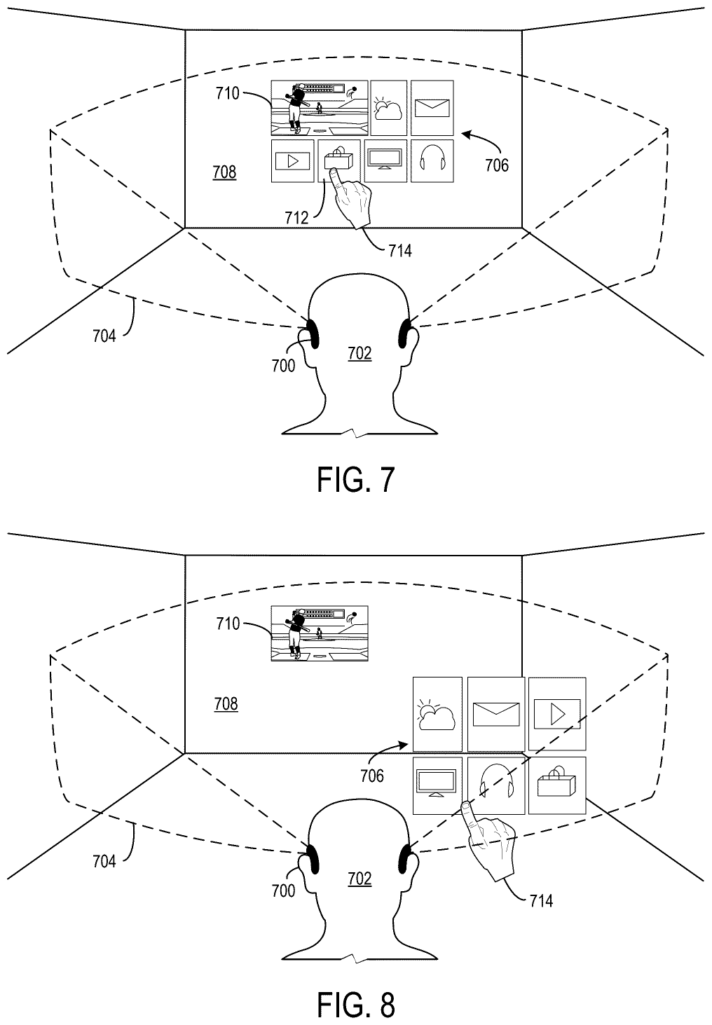

In some implementations, the GUI optimizer 224 may optimize a 3D GUI including a plurality of user interface objects each having 3D locations based at least on the activation data 220. FIGS. 7-8 shows an example scenario in which a 3D GUI is optimized based at least on activation data generated from a user interacting with the 3D GUI. In FIG. 7, an augmented-reality device 700 is worn by a user 702. The augmented-reality device 700 visually presents a 3D GUI 704 including a plurality of user interface objects 706 that appear to be incorporated into a real-world physical space 708 surrounding a real-world, physical television 710. The user 702 interacts with the plurality of user interface objects 706 using hand gestures. In particular, the user 702 selects a user interface object 712 by reaching out and pointing with a right hand 714. Activation data that correlates an amount of muscle activation of the user's right hand 714 to a location of the user's right hand 714 in the physical space 708 is generated based at least on the user 702 interacting with the 3D GUI 704.

In FIG. 8, the 3D GUI 704 is optimized based at least on the activation data to reduce muscle activation of the user's right hand 714 while the user 702 is interacting with the 3D GUI 704. In particular, the plurality of user interface objects 706 are positioned in the 3D GUI 704 closer to the user's right hand 714, so that the user 702 does not have to reach as far into the physical space 708 to interact with the plurality of user interface objects 706. By not having to reach as far to interact with the plurality of user interface objects 706, a degree of muscle activation of the user's right hand 714 (and arm and shoulder) may be reduced relative to interacting with the arrangement of user interface objects shown in the 3D GUI in FIG. 7.

The example scenario shown in FIGS. 7-8 is meant to be non-limiting. The plurality of user interface objects 706 may be arranged in the 3D GUI 704 in any suitable manner based at least on the activation data.

Returning to FIG. 2, in some implementations, the GUI optimizer 224 may be configured to set a default arrangement 230 of the plurality of user interface objects 226 in the GUI 228 based at least on the activation data 220. For example, the activation data 220 may be used in a research and development scenario in order to design the default arrangement 230 of the plurality of user interface objects 226 in the GUI 228 to reduce an amount of muscle activation while an anticipated user is interacting with the GUI 228. In some examples, one or more of a size and a location of the plurality of user interface objects 226 may be set in the default arrangement 230 based at least on the activation data 220 to reduce or minimize muscle activation of body part(s) while interacting with the GUI 228. Such a reduction or minimization of muscle activation while interacting with the GUI improves human-computer interaction and reduces the burden of user input to a computing device, since a user can make low-strain movements more quickly and more frequently with less fatigue relative to interacting with other GUIs that are arranged using other methods that do not consider reducing muscle activation/strain/effort.

In some implementations, the activation data 220 may be aggregated for a plurality of users having different physical characteristics (e.g., different hand size, arm size, leg size), and the GUI optimizer 224 may be configured to design the default arrangement 230 of the plurality of user interface objects 226 in the GUI 228, such that the default arrangement 230 is optimized collectively for the plurality of users.

In some implementations, the GUI optimizer 224 may be configured to design a plurality of different default arrangements of the plurality of user interface objects 226 having different sizes and/or positions in the GUI 228. The plurality of different default arrangements may be designed to optimize the GUI 228 for different groups of users having different body part characteristics and the different default arrangements may be optimized differently to accommodate the different body part characteristics.

In one example, the GUI optimizer 224 may generate a first default arrangement of user interface objects based at least on activation data for a first group of users that are characterized by having smaller sized hands (e.g., smaller fingers and/or a smaller range of movement). Further, the GUI optimizer 224 may generate a second default arrangement of user interface objects based at least on activation data for a second group of users that are characterized by having larger sized hands (e.g., larger fingers and/or a larger range of movement). For example, the first default arrangement may have smaller sized user interface objects arranged in a tighter grouping, so that the users with smaller hands can reach the different user interface objects more easily with less muscle activation. Further, the second default arrangement may have larger sized user interface objects that are spaced apart more, since the users with larger hands can reach further relative to the user with smaller hands without additional muscle strain/effort.

In some implementations, users may perform a calibration routine that allows for body part characteristics to be assessed in order for a suitable default arrangement to be selected. In some examples, the calibration routine may generate activation data that is used for the assessment. In some examples, the calibration routine may include a user explicitly providing body part characteristic information to select a default arrangement for the GUI.

In some implementations where the GUI optimizer 224 is configured to arrange the plurality of user interface objects 226 in the default arrangement 230 in the GUI 228, the GUI optimizer 224 may output the GUI 228 as instructions that are incorporated in an application program 232 that may be executed to visually present the GUI 228. In some examples, the computing system 200 may execute the application program 232 to visually present the GUI 228 via a display 234 communicatively coupled with the computing system 200. In other examples, the application program 232 may be sent to one or more network computers 236 via a computer network 238. Further, the one or more network computers 236 may executed the application program 232 to visually present the GUI 228 including the default arrangement 230 of user interface objects 226 via a local display associated with the one or more network computers 236. In other words, the computing system 200 may be configured to design an optimized GUI of user interface objects offline based at least on aggregated activation data and the GUI may be used in the application program that is executed by other computers. In this scenario, the computing system that is generating the activation data 220 based at least on the muscle activation signal(s) 204 and the spatial signal(s) 214 and optimizing the GUI 228 need not be the same computing system that is visually presenting the GUI 228. However, in some implementations, the same computing system may generate the optimized GUI 228 and visually present the GUI 228 via the associated display 234.

In some implementations, the GUI optimizer 224 may be configured to dynamically customize the GUI 228 for a designated user based at least on activation data generated for the user while the user is interacting with the GUI 228. In particular, the computing system 200 may be configured to visually present, via the display 234, the GUI 228 including the default arrangement 230 of the plurality of user interface objects 226. In some examples, the default arrangement 230 may be optimized for a population of users based at least on activation data previously collected for the population of users. In other examples, the default arrangement 230 may be designed based at least on other factors that do not consider activation data.

The spatial muscle activity tracker 202 is configured to receive muscle activation signal(s) 204 and spatial signal(s) 214 corresponding to one or more body parts 212 of a user while the GUI 228 is being visually presented and the user is interacting with the GUI 228. The spatial muscle activity tracker 202 is configured to generate activation data 220 based at least on the muscle activation signal(s) 204 and spatial signal(s) 214.

The GUI optimizer 224 is configured to dynamically adjust the default arrangement 230 of the plurality of user interface objects 226 to a customized arrangement 240 of the plurality of user interface objects in the GUI based at least on the activation data 220. The plurality of user interface objects 226 may be dynamically adjusted in the customized arrangement 240 in any suitable manner. In some examples, a size of one or more of the user interface objects 226 may be dynamically adjusted based at least on the activation data 220. In some examples, a location of one or more of the user interface objects may be dynamically adjusted based at least on the activation data 220. For example, one or more user interface object may be dynamically adjusted to reduce muscle activation of the user's body part while the user is interacting with the GUI 228. Further, the computing system 200 is configured to visually present, via the display 234, the GUI 228 including the customized arrangement 240 of a plurality of user interface objects 226.

In some examples, the GUI optimizer 224 may be configured to dynamically adjust an arrangement of 2D user interface objects in a 2D GUI while a user is interacting with the 2D GUI. Returning to the example of the virtual keyboard, the GUI optimizer 224 may be configured to dynamically adjusting a default arrangement of the plurality of virtual keys of the virtual keyboard (e.g., shown in FIG. 5) to a customized arrangement of the plurality of virtual keys of (e.g., shown in FIG. 6) based at least on the activation data generated while the user is interacting with the virtual keyboard via touch input.

In some examples, the GUI optimizer 224 may be configured to dynamically adjust an arrangement of 3D user interface objects in a 3D GUI while a user is interacting with the 3D GUI. Returning to the example of the 3D GUI visually presented by the augmented-reality device, the GUI optimizer 224 may be configured to dynamically adjusting a default arrangement (e.g., shown in FIG. 7) of the plurality of 3D user interface objects in the physical space to a customized arrangement of the plurality of 3D user interface objects (e.g., shown in FIG. 8) based at least on the activation data generated while the user is interacting with the 3D GUI via hand gestures.

In some implementations, at least some of the functionality of the computing system 200 may be performed by the network computer(s) 236. In one example, the computing system 200 may be configured to output the activation data 220 to the network computer(s) 236 and the network computer(s) 236 may be configured to optimize the GUI 228 based at least on the activation data 220. In other examples, the network computer(s) 236 may be configured to output the activation data 220 to the computing system 200 and the computing system 200 may be configured to optimize the GUI 228 based at least on the activation data 220.

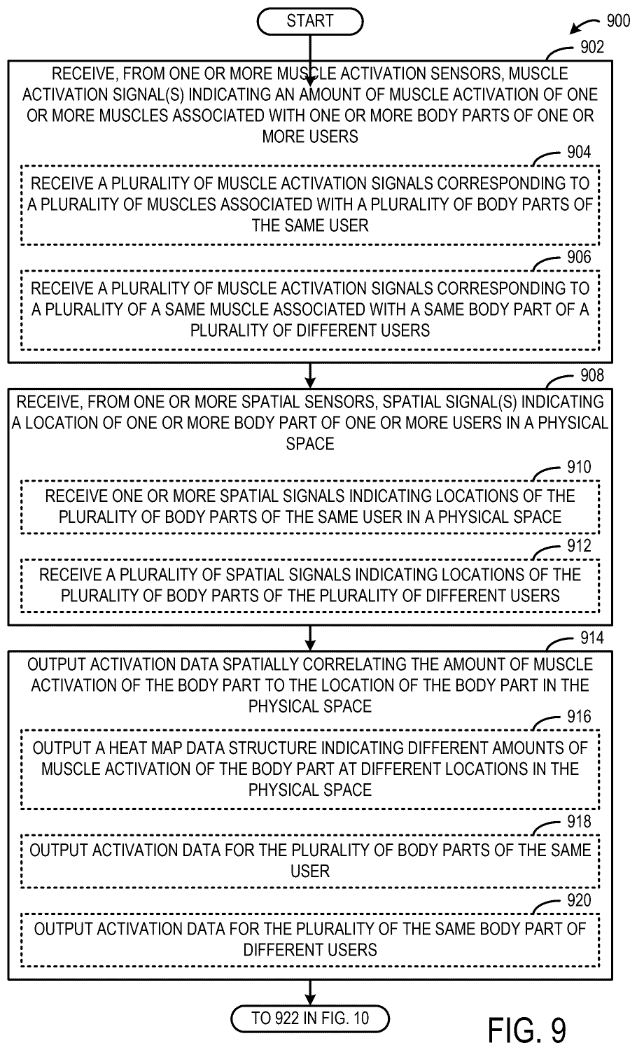

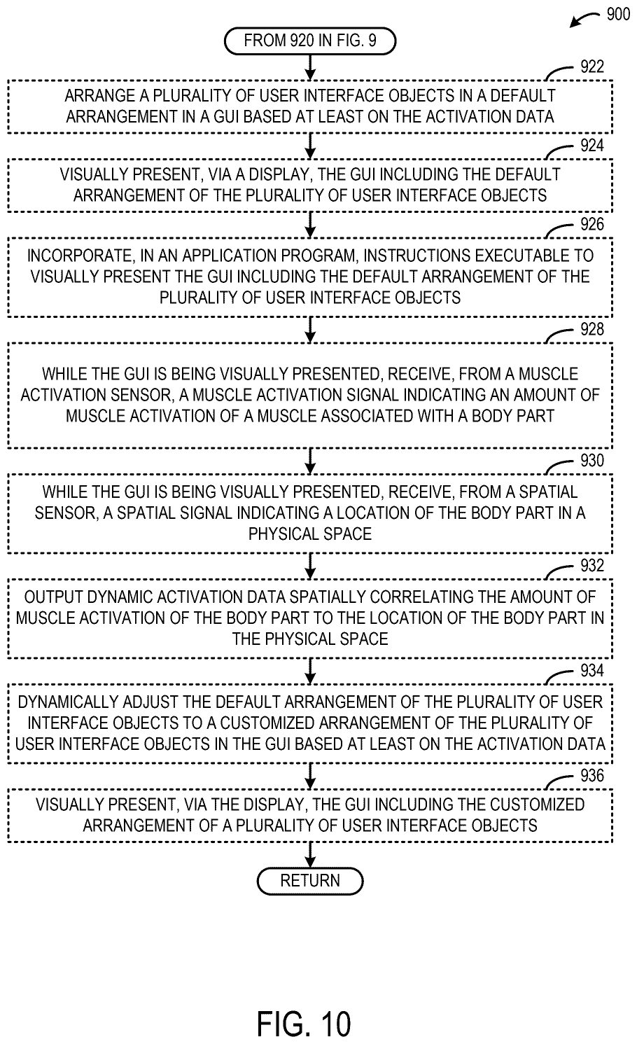

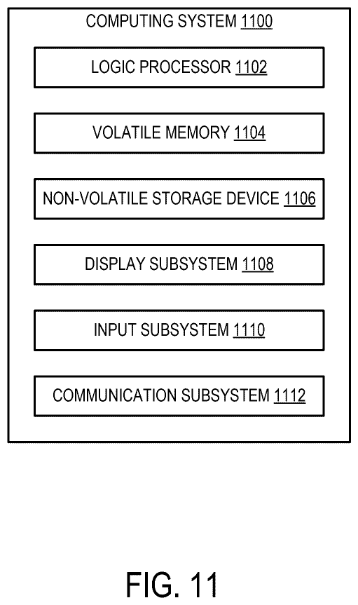

FIGS. 9-10 show an example computer-implemented method 900 for spatially tracking muscle activity of one or more users. For example, the computer-implemented method 900 may be performed by any of the computing devices 100A, 100B, 100C shown in FIG. 1, the computing system 200 and/or the network computer(s) 236 shown in FIG. 2, and/or the computing system 1100 shown in FIG. 11.

In FIG. 9, at 902, the computer-implemented method 900 includes receiving, from one or more muscle activation sensors, muscle activation signal(s) indicating an amount of muscle activation of one or more muscles associated with one or more body parts of one or more users.

In some implementations, at 904, the computer-implemented method 900 may include receiving a plurality of muscle activation signals corresponding to a plurality of muscles associated with a plurality of body parts of a same user.

In some implementations, at 906, the computer-implemented method 900 may include receiving a plurality of muscle activation signals corresponding to a plurality of the same muscle associated with a same body part of a plurality of different users.

At 908, the computer-implemented method 900 includes receiving, from one or more spatial sensors, spatial signal(s) indicating a location of one or more body parts of one or more users in a physical space.

In some implementations where a plurality of different body parts of a same user is tracked, at 910, the computer-implemented method 900 may include receiving one or more spatial signals indicating locations of the plurality of body parts of the same user in the physical space.

In some implementations where a plurality of the same body part of different users is tracked, at 912, the computer-implemented method 900 may include receiving a plurality of spatial signals indication location of the plurality of the same body part of the different users.

At 914, the computer-implemented method 900 includes outputting activation data spatially correlating the amount of muscle activation of the body part to the location of the body part in the physical space.

In some implementations, at 916, the computer-implemented method 900 may include outputting a heat map data structure indicating different amounts of muscle activation of the body part at different locations in the physical space.

In some implementations where a plurality of different body parts of a same user is tracked, at 918, the computer-implemented method 900 may include outputting activation data for the plurality of different body parts of the same user.

In some implementations where a plurality of the same body part of different users is tracked, at 920, the computer-implemented method 900 may include outputting activation data for the plurality of same body parts of the different users.

In FIG. 10, in some implementations, at 922, the computer-implemented method 900 may include arranging a plurality of user interface objects in a default arrangement in a GUI based at least on the activation data. In some implementations, the default arrangement may be optimized to reduce muscle activation of body parts that interact with the user interface objects in the default arrangement of the GUI. In some implementations, the default arrangement may be optimized for one or more body parts of the same user. In some implementations, the default arrangement may be optimized for a plurality of different users.

In some implementations, at 924, the computer-implemented method 900 may include visually presenting, via a display, a GUI including the default arrangement of the plurality of user interface objects.

In some implementations, at 926, the computer-implemented method 900 may include incorporating, in an application program, instructions executable to visually present the GUI including the default arrangement of the plurality of user interface objects. For example, the application program may be executed by different computers associated with different users to allow the different users to interact with the GUI including the default arrangement of user interface objects.

In some implementations, at 928, the computer-implemented method 900 may include while the GUI is being visually presented, receiving, from a muscle activation sensor, a muscle activation signal indicating an amount of muscle activation of a muscle associated with a body part.

In some implementations, at 930, the computer-implemented method 900 may include receiving, from a spatial sensor, a spatial signal indicating a location of the body part in a physical space.

In some implementations, at 932, the computer-implemented method 900 may include outputting dynamic activation data spatially correlating the amount of muscle activation of the body part to the location of the body part in the physical space.

In some implementations, at 934, the computer-implemented method 900 may include dynamically adjusting the default arrangement of the plurality of user interface objects to a customized arrangement of the plurality of user interface objects in the GUI based at least on the activation data.

In some implementations, at 934, the computer-implemented method 900 may include visually presenting, via the display, the GUI including the customized arrangement of the plurality of user interface objects.

The computer-implemented method may be performed to spatially track muscle activity of one or more users in the form of activation data that spatially correlates an amount of muscle activation of a body part to a location of the body part in a physical space. Such activation data can be used to provide an accurate assessment of movement efficiency of the body part in the physical space.

Furthermore, the activation data can be leveraged to optimize an arrangement of user interface objects in a GUI in terms of minimizing muscle activation/strain/effort, joint strain, and/or improving movement efficiency. By optimizing an arrangement of user interface objects in a GUI based at least on the activation data, in the short-term, a user's muscle activation/strain/effort may be reduced while interacting with the GUI relative to interacting with another GUI arranged in a different manner that does not consider muscle activation. Moreover, a likelihood of long-term overuse injuries from interacting with the GUI may be reduced relative to interacting with another GUI arranged in a different manner that does not consider muscle activation. Further, optimizing the GUI in terms of user movement efficiency based at least on the activation data may increase user productivity, since a user can make low-strain movements more quickly and more frequently with less fatigue relative to interacting with other GUIs that are arranged using other methods that do not consider minimizing muscle activation/strain/effort.

In some implementations, the GUI may be optimized for a plurality of different body parts of the same user based on activation data generated for the plurality of different body parts. In some implementations, the GUI may be designed in a research and development scenario where activation data is generated for a plurality of different users and the GUI is optimized collectively for the plurality of different users. In some implementations, the GUI may be dynamically customized for a user based at least one activation data that is generated while the user is interacting with the GUI.

In some implementations, the methods and processes described herein may be tied to a computing system of one or more computing devices. In particular, such methods and processes may be implemented as a computer-application program or service, an application-programming interface (API), a library, and/or other computer-program product.

FIG. 11 schematically shows a non-limiting implementation of a computing system 1100 that can enact one or more of the methods and processes described above. Computing system 1100 is shown in simplified form. Computing system 1100 may embody any of the computing devices 100A-100C shown in FIG. 1, the computing system 200 shown in FIG. 2, and/or the network computer(s) 236 shown in FIG. 2. Computing system 1100 may take the form of one or more personal computers, server computers, tablet computers, home-entertainment computers, network computing devices, gaming devices, mobile computing devices, mobile communication devices (e.g., smart phone), and/or other computing devices, and wearable computing devices such as smart wristwatches, backpack host computers, and head-mounted augmented/mixed virtual reality devices.

Computing system 1100 includes a logic processor 1102, volatile memory 1104, and a non-volatile storage device 1106. Computing system 1100 may optionally include a display subsystem 1108, input subsystem 1110, communication subsystem 1112, and/or other components not shown in FIG. 11.

Logic processor 1102 includes one or more physical devices configured to execute instructions. For example, the logic processor may be configured to execute instructions that are part of one or more applications, programs, routines, libraries, objects, components, data structures, or other logical constructs. Such instructions may be implemented to perform a task, implement a data type, transform the state of one or more components, achieve a technical effect, or otherwise arrive at a desired result.

The logic processor 1102 may include one or more physical processors (hardware) configured to execute software instructions. Additionally or alternatively, the logic processor may include one or more hardware logic circuits or firmware devices configured to execute hardware-implemented logic or firmware instructions. Processors of the logic processor 1102 may be single-core or multi-core, and the instructions executed thereon may be configured for sequential, parallel, and/or distributed processing. Individual components of the logic processor optionally may be distributed among two or more separate devices, which may be remotely located and/or configured for coordinated processing. Aspects of the logic processor may be virtualized and executed by remotely accessible, networked computing devices configured in a cloud-computing configuration. In such a case, these virtualized aspects are run on different physical logic processors of various different machines, it will be understood.

Non-volatile storage device 1106 includes one or more physical devices configured to hold instructions executable by the logic processors to implement the methods and processes described herein. When such methods and processes are implemented, the state of non-volatile storage device 1106 may be transformed—e.g., to hold different data.

Non-volatile storage device 1106 may include physical devices that are removable and/or built-in. Non-volatile storage device 1106 may include optical memory (e.g., CD, DVD, HD-DVD, Blu-Ray Disc, etc.), semiconductor memory (e.g., ROM, EPROM, EEPROM, FLASH memory, etc.), and/or magnetic memory (e.g., hard-disk drive, floppy-disk drive, tape drive, MRAM, etc.), or other mass storage device technology. Non-volatile storage device 1106 may include nonvolatile, dynamic, static, read/write, read-only, sequential-access, location-addressable, file-addressable, and/or content-addressable devices. It will be appreciated that non-volatile storage device 1106 is configured to hold instructions even when power is cut to the non-volatile storage device 1106.

Volatile memory 1104 may include physical devices that include random access memory. Volatile memory 1104 is typically utilized by logic processor 1102 to temporarily store information during processing of software instructions. It will be appreciated that volatile memory 1104 typically does not continue to store instructions when power is cut to the volatile memory 1104.

Aspects of logic processor 1102, volatile memory 1104, and non-volatile storage device 1106 may be integrated together into one or more hardware-logic components. Such hardware-logic components may include field-programmable gate arrays (FPGAs), program- and application-specific integrated circuits (PASIC/ASICs), program- and application-specific standard products (PSSP/ASSPs), system-on-a-chip (SOC), and complex programmable logic devices (CPLDs), for example.

The spatial muscle activity tracker 202 and the GUI optimizer 224 describe aspects of the computing system 200 implemented to perform particular functions. In some cases, the spatial muscle activity tracker 202 and the GUI optimizer 224 may be instantiated via logic machine 1102 executing instructions held by storage machine 1106. It will be understood that the spatial muscle activity tracker 202 and the GUI optimizer 224 as well as any other modules, programs, and/or engines may be instantiated from the same application, service, code block, object, library, routine, API, function, etc. Likewise, the spatial muscle activity tracker 202 and the GUI optimizer 224, or a same module, program, and/or engine may be instantiated by different applications, services, code blocks, objects, routines, APIs, functions, etc. The terms “module,” “program,” and “engine” may encompass individual or groups of executable files, data files, libraries, drivers, scripts, database records, etc.

When included, display subsystem 1108 may be used to present a visual representation of data held by non-volatile storage device 1106. The visual representation may take the form of a graphical user interface (GUI). As the herein described methods and processes change the data held by the non-volatile storage device, and thus transform the state of the non-volatile storage device, the state of display subsystem 1108 may likewise be transformed to visually represent changes in the underlying data. Display subsystem 1108 may include one or more display devices utilizing virtually any type of technology. Such display devices may be combined with logic processor 1102, volatile memory 1104, and/or non-volatile storage device 1106 in a shared enclosure, or such display devices may be peripheral display devices.

When included, input subsystem 1110 may comprise or interface with one or more user-input devices such as a keyboard, mouse, touch screen, microphone for speech and/or voice recognition, a camera (e.g., a webcam), or game controller.

When included, communication subsystem 1112 may be configured to communicatively couple various computing devices described herein with each other, and with other devices. Communication subsystem 1112 may include wired and/or wireless communication devices compatible with one or more different communication protocols. As non-limiting examples, the communication subsystem may be configured for communication via a wireless telephone network, or a wired or wireless local- or wide-area network, such as a HDMI over Wi-Fi connection. In some implementations, the communication subsystem may allow computing system 1100 to send and/or receive messages to and/or from other devices via a network such as the Internet.

In an example, a computer-implemented method for spatially tracking muscle activity comprises receiving, from a muscle activation sensor, a muscle activation signal indicating an amount of muscle activation of a muscle associated with a body part, receiving, from a spatial sensor, a spatial signal indicating a location of the body part in a physical space, and outputting activation data spatially correlating the amount of muscle activation of the body part to the location of the body part in the physical space. In this example and/or other examples, the activation data may be output as a heat map data structure indicating different amounts of muscle activation of the body part at different locations in the physical space. In this example and/or other examples, the computer-implemented method may further comprise receiving, from a plurality of muscle activation sensors associated with a same user, a plurality of muscle activation signals corresponding to a plurality of muscles associated with a plurality of body parts of the same user, each muscle activation signal indicating an amount of muscle activation of a corresponding muscle of the plurality of muscles, receiving, from one or more spatial sensors, one or more spatial signals indicating locations of the plurality of body parts in a physical space, and the activation data may spatially correlate the amount of muscle activation of each of the plurality of body parts to each of the locations of the plurality of body parts in the physical space. In this example and/or other examples, the computer-implemented method may further comprise receiving, from a plurality of muscle activation sensors associated with a plurality of different users, a plurality of muscle activation signals corresponding to a plurality of a same muscle associated with a same body part of the plurality of different users, each muscle activation signal indicating an amount of muscle activation of a corresponding muscle of the plurality of the same muscles, receiving, from one or more spatial sensors, a plurality of spatial signals indicating locations of the plurality of body parts of the plurality of different users, and the activation data may spatially correlate the amount of muscle activation of each of the plurality body parts of the plurality of different users to each of the locations of the plurality of body parts. In this example and/or other examples, the computer-implemented method may further comprise visually presenting, via a display, a graphical user interface including a plurality of user interface objects arranged in the graphical user interface based at least on the activation data. In this example and/or other examples, a size of a user interface object of the plurality of user interface objects may be set based at least on the activation data. In this example and/or other examples, a location of a user interface object of the plurality of user interface objects in the graphical user interface may be set based at least on the activation data. In this example and/or other examples, the graphical user interface may be a two-dimensional, 2D, graphical user interface, the plurality of user interface objects may each have a 2D location in the 2D graphical user interface, and the location of the body part may be mapped to a 2D location in the 2D graphical user interface. In this example and/or other examples, the graphical user interface may be a three-dimensional, 3D, graphical user interface, the plurality of user interface objects may each have a 3D location in the 3D graphical user interface, and the location of the body part may be mapped to a 3D location in the 3D graphical user interface. In this example and/or other examples, the computer-implemented method may further comprise tracking interaction of the body part with the plurality of user interface objects, a more-frequently-used user interface object of the plurality of user interface objects having a higher interaction frequency with the body part over the period of time may be positioned in the graphical user interface at a location correlated with a smaller amount of muscle activation based at least on the activation data, and a less-frequently-used user interface object of the plurality of user interface objects having a lower interaction frequency over the period of time may be positioned in the graphical user interface at a location correlated with a larger amount of muscle activation based at least on the activation data. In this example and/or other examples, the computer-implemented method may further comprise dynamically adjusting a default arrangement of the plurality of user interface objects to a customized arrangement of the plurality of user interface objects in a graphical user interface based at least on the activation data, and visually presenting, via the display, the graphical user interface including the customized arrangement of a plurality of user interface objects. In this example and/or other examples, one or more of a size of a user interface object and location of the user interface object in the graphical user interface may dynamically adjusted based at least on the activation data. In this example and/or other examples, the one or more muscle activation sensors may include an electromyography, EMG, sensor. In this example and/or other examples, the one or more spatial sensors may include a camera. In this example and/or other examples, the one or more spatial sensors may include a touch sensor of a touch-sensitive display device.

In another example, a computer-implemented method for spatially tracking muscle activity comprises receiving, from a plurality of muscle activation sensors associated with a plurality of different users, a plurality of muscle activation signals corresponding to a plurality of a same muscle associated with a same body part of the plurality of different users, each muscle activation signal indicating an amount of muscle activation of a corresponding muscle of the plurality of the same muscles, receiving, from one or more spatial sensors, a plurality of spatial signals indicating locations of the plurality of body parts of the plurality of different users, and outputting activation data spatially correlating the amount of muscle activation of each of the plurality body parts of the plurality of different users to each of the locations of the plurality of body parts. In this example and/or other examples, the activation data may be output as a heat map data structure indicating different amounts of muscle activation of the body part at different locations in the physical space. In this example and/or other examples, the computer-implemented method may further comprise arranging a plurality of user interface objects in a graphical user interface based at least on the activation data. In this example and/or other examples, one or more of a size and a location of a user interface object of the plurality of user interface objects may be set based at least on the activation data.

In yet another example, a computing system comprises a logic processor, and a storage device holding instructions executable by the logic processor to carry out any of the above-described examples of computer-implemented methods.

It will be understood that the configurations and/or approaches described herein are exemplary in nature, and that these specific embodiments or examples are not to be considered in a limiting sense, because numerous variations are possible. The specific routines or methods described herein may represent one or more of any number of processing strategies. As such, various acts illustrated and/or described may be performed in the sequence illustrated and/or described, in other sequences, in parallel, or omitted. Likewise, the order of the above-described processes may be changed.

The subject matter of the present disclosure includes all novel and non-obvious combinations and sub-combinations of the various processes, systems and configurations, and other features, functions, acts, and/or properties disclosed herein, as well as any and all equivalents thereof.

1. A computer-implemented method for spatially tracking muscle activity, the method comprising:
receiving, from a muscle activation sensor, a muscle activation signal indicating an amount of muscle activation of a muscle associated with a body part;
receiving, from a spatial sensor, a spatial signal indicating a location of the body part in a physical space; and
outputting activation data spatially correlating the amount of muscle activation of the body part to the location of the body part in the physical space; and
visually presenting, via a display, a graphical user interface including a plurality of user interface objects arranged in the graphical user interface based at least on the activation data.

2. The computer-implemented method of claim 1, wherein the activation data is output as a heat map data structure indicating different amounts of muscle activation of the body part at different locations in the physical space.

3. The computer-implemented method of claim 1, further comprising:
receiving, from a plurality of muscle activation sensors associated with a same user, a plurality of muscle activation signals corresponding to a plurality of muscles associated with a plurality of body parts of the same user, each muscle activation signal indicating an amount of muscle activation of a corresponding muscle of the plurality of muscles;
receiving, from one or more spatial sensors, one or more spatial signals indicating locations of the plurality of body parts in a physical space; and
wherein the activation data spatially correlates the amount of muscle activation of each of the plurality of body parts to each of the locations of the plurality of body parts in the physical space.

4. The computer-implemented method of claim 1, further comprising:
receiving, from a plurality of muscle activation sensors associated with a plurality of different users, a plurality of muscle activation signals corresponding to a plurality of a same muscle associated with a same body part of the plurality of different users, each muscle activation signal indicating an amount of muscle activation of a corresponding muscle of the plurality of the same muscles;
receiving, from one or more spatial sensors, a plurality of spatial signals indicating locations of the plurality of body parts of the plurality of different users; and
wherein the activation data spatially correlates the amount of muscle activation of each of the plurality body parts of the plurality of different users to each of the locations of the plurality of body parts.

5. The computer-implemented method of claim 1, wherein a size of a user interface object of the plurality of user interface objects is set based at least on the activation data.

6. The computer-implemented method of claim 1, wherein a location of a user interface object of the plurality of user interface objects in the graphical user interface is set based at least on the activation data.

7. The computer-implemented method of claim 1, wherein the graphical user interface is a two-dimensional, 2D, graphical user interface, wherein the plurality of user interface objects each have a 2D location in the 2D graphical user interface, and wherein the location of the body part is mapped to a 2D location in the 2D graphical user interface.

8. The computer-implemented method of claim 1, wherein the graphical user interface is a three-dimensional, 3D, graphical user interface, wherein the plurality of user interface objects each have a 3D location in the 3D graphical user interface, and wherein the location of the body part is mapped to a 3D location in the 3D graphical user interface.

9. The computer-implemented method of claim 1, further comprising:
tracking interaction of the body part with the plurality of user interface objects;
wherein a more-frequently-used user interface object of the plurality of user interface objects having a higher interaction frequency with the body part over the period of time is positioned in the graphical user interface at a location correlated with a smaller amount of muscle activation based at least on the activation data, and
wherein a less-frequently-used user interface object of the plurality of user interface objects having a lower interaction frequency over the period of time is positioned in the graphical user interface at a location correlated with a larger amount of muscle activation based at least on the activation data.

10. The computer-implemented method of claim 1, further comprising:
dynamically adjusting a default arrangement of the plurality of user interface objects to a customized arrangement of the plurality of user interface objects in a graphical user interface based at least on the activation data; and
visually presenting, via the display, the graphical user interface including the customized arrangement of a plurality of user interface objects.

11. The computer-implemented method of claim 10, wherein one or more of a size of a user interface object and location of the user interface object in the graphical user interface is dynamically adjusted based at least on the activation data.

12. The computer-implemented method of claim 1, wherein the muscle activation sensor includes an electromyography, EMG, sensor.

13. The computer-implemented method of claim 1, wherein the spatial sensor includes a camera.

14. The computer-implemented method of claim 1, wherein the spatial sensor includes a touch sensor of a touch-sensitive display device.

15. A computer-implemented method for spatially tracking muscle activity, the computer-implemented method comprising:
receiving, from a plurality of muscle activation sensors associated with a plurality of different users, a plurality of muscle activation signals corresponding to a plurality of a same muscle associated with a same body part of the plurality of different users, each muscle activation signal indicating an amount of muscle activation of a corresponding muscle of the plurality of the same muscles;
receiving, from one or more spatial sensors, a plurality of spatial signals indicating locations of the plurality of body parts of the plurality of different users; and
outputting activation data spatially correlating the amount of muscle activation of each of the plurality body parts of the plurality of different users to each of the locations of the plurality of body parts; and
arranging a plurality of user interface objects in a graphical user interface based at least on the activation data.

16. The computer-implemented method of claim 16, wherein the activation data is output as a heat map data structure indicating different amounts of muscle activation of the body part at different locations in the physical space.

17. The computer-implemented method of claim 16, wherein one or more of a size and a location of a user interface object of the plurality of user interface objects is set based at least on the activation data.

18. A computing system comprising:
a logic processor; and
a storage device holding instructions executable by the logic processor to
receive, from a muscle activation sensor, a muscle activation signal indicating an amount of muscle activation of a muscle associated with a body part;
receive, from a spatial sensor, a spatial signal indicating a location of the body part in a physical space; and
output activation data spatially correlating the amount of muscle activation of the body part to the location of the body part in the physical space; and
visually present, via a display, a graphical user interface including a plurality of user interface objects arranged in the graphical user interface based at least on the activation data.

19. The computing system of claim 18, wherein the activation data is output as a heat map data structure indicating different amounts of muscle activation of the body part at different locations in the physical space.

20. The computing system of claim 18, wherein the storage device holds instructions executable by the logic processor to:
receive, from a plurality of muscle activation sensors associated with a same user, a plurality of muscle activation signals corresponding to a plurality of muscles associated with a plurality of body parts of the same user, each muscle activation signal indicating an amount of muscle activation of a corresponding muscle of the plurality of muscles;
receive, from one or more spatial sensors, one or more spatial signals indicating locations of the plurality of body parts in a physical space; and
wherein the activation data spatially correlates the amount of muscle activation of each of the plurality of body parts to each of the locations of the plurality of body parts in the physical space.
